(19) United States
(12) Patent Application Publication
(10) Pub. No.: US 2010/0098759 A1
(43) Pub. Date: Apr. 22, 2010

(54) CONTROLLED-RELEASE PREPARATION CONTAINING CILOSTAZOL AND PROCESS FOR THE PREPARATION THEREOF

(75) Inventors: Jin Woo Park, Gangseo-gu (KR); Kwang Hyun Shin, Paldal-gu (KR); Sung Ah Bin, Giheung-gu (KR); Hyeok Lee, Bundang-gu (KR); Joon Ho Bae, Suji-gu (KR); Do Yong Jeon, Dobong-gu (KR)

Correspondence Address: LAW OFFICES OF KHALILIAN SIRA, LLC, 9100 PERSIMMON TREE ROAD, POTOMAC, MD, 20854, US

(73) Assignees: AMOREPACIFIC CORPORATION;, Seoul (KR); PACIFIC PHARMACEUTICAL CO., LTD., Seoul (KR)

(21) Appl. No.: 12/527,027

(22) PCT Filed: Feb. 15, 2008
(86) PCT No.: PCT/KR2008/000902

§ 371 Date: Aug. 13, 2009

(30) Foreign Application Priority Data
Feb. 15, 2007 (KR) 10-2007-0016221

(51) Int. Cl. A61K 9/28; A61K 31/4709

(52) U.S. Cl. 424/474; 514/312

(57) ABSTRACT

The present invention relates to a controlled-release formulation comprising cilostazol and a method for preparing said formulation. The inventive controlled-release formulation comprising cilostazol or a pharmaceutically acceptable salt thereof, a solubilizing agent, a swelling agent, a swell-controlling agent and a gas generating material has advantages in that it maintains a constant cilostazol level in the blood through a slow release while it resides in the stomach and intestines over a long period of time, thereby increasing the absorption of cilostazol in the small intestine, the major absorption site of cilostazol, as well as minimizing adverse effects caused by rapid release and making it easy for a patient to take the drug.

### FIELD OF THE INVENTION

The present invention relates to a controlled-release formulation comprising cilostazol and a method for preparing said formulation.

### BACKGROUND OF THE INVENTION

Cilostazol is a representative intracellular cAMP PDE (cyclic AMP phosphodiesterase) inhibitor and has been known to play significant roles in the suppression of the blood coagulation, the promotion of the central blood circulation, anti-inflammation and anti-ulcer actions, the prevention and treatment of asthma and cerebral infarction, and the improvement of the cerebral circulation, by decreasing platelet coagulation and dilating the arteries through the inhibition of PDE activity.

Cilostazol has a poor water-solubility (1 μg/ml or less), and it has been demonstrated that orally administered cilostazol is absorbed mainly in the upper gastrointestinal (GI) tract and the absorption thereof is reduced as it moves to the lower GI tract. Therefore, the currently available preparations of cilostazol are of the form of a rapid release tablet because there is a limit to the absorption time in a controlled-release formulation of cilostazol. However, such a rapid release formulation of cilostazol can induce a sudden elevation of the drug concentration in the blood when orally administered, which results in adverse effects such as headache, and the dosage thereof is inconvenient in that the rapid release formulation should be administered twice a day in an amount ranging from 50 to 100 mg in order to maintain their constant pharmacological activities.

Accordingly, there have been numerous attempts to develop a sustained or controlled-release formulation of cilostazol that is free from the above problems.

For example, International Patent Publication No. WO 97/48382 discloses a sustained-release formulation of cilostazol of a multiple unit form comprising at least 2 mini tablets using hydroxypropylmethylcellulose as a main matrix, and International Patent Publication No. WO 96/21448 discloses a sustained release formulation of a resin particle form having a particle size less than 2,000 μm in diameter, which comprises cilostazol and ethylene-vinyl alcohol copolymer. However, these sustained release formulations exhibit ineffective drug absorption since poorly soluble cilostazol having restricted absorption sites is released too slowly. To solve this problem, International Patent Publication No. WO 00/57881 and U.S. Patent Publication No. 2002/0058066 have suggested a formulation comprising an external layer and a nucleus, the external layer slowly releasing the drug in the upper GI tract (the small intestine) and the nucleus releasing the drug rapidly in the lower small intestine and the colon. However, this formulation has the problems that: the rapid release of the drug in the lower small intestine and the colon can cause mucosal damage; the absorption rate of the poorly soluble drug becomes irregular in the lower intestine and colon where the water content is relatively low; the process for the preparation thereof is very complicated; and the large volume of the daily dosage of the formulation makes it difficult for a patient to take the drug.

### SUMMARY OF THE INVENTION

Accordingly, it is an object of the present invention to provide an improved controlled-release formulation containing cilostazol or a pharmaceutically acceptable salt thereof.

It is another object of the present invention to provide a method for preparing said formulation.

In accordance with one aspect of the present invention, there is provided a controlled-release formulation comprising cilostazol or a pharmaceutically acceptable salt thereof, a solubilizing agent, a swelling agent, a swell-controlling agent, and a gas generating material.

In accordance with another aspect of the present invention, there is provided a method for preparing said controlled-release formulation comprising the steps of:

1) mixing cilostazol or a pharmaceutically acceptable salt thereof, a solubilizing agent, a swelling agent, a swell-controlling agent, and a gas generating material, granulating the resulting mixture; and

2) formulating the resulting mixture in the form of a capsule or a tablet.

### DETAILED DESCRIPTION OF THE INVENTION

The controlled-release formulation of the present invention is designed to swell and float in the upper GI tract, i.e., the stomach and intestines, so as to extend its retention time therein, which is made possible by the combined action of a swelling agent, a swell-controlling agent, and a gas generating material, and also to maintain an effective drug release rate at its absorption sites located in the small intestine by the action of a solubilizing agent which prevents a delay in the drug release caused by using the swelling agent.

Accordingly, the controlled-release formulation of the present invention has advantages in that it maintains a constant drug level of cilostazol in blood through a slow release while it resides in the stomach and intestines over a long period of time, thereby increasing the absorption of cilostazol in the small intestine known as a major absorption site of cilostazol as well as minimizing adverse effects caused by rapid release and enhancing the patient compliance.

Hereinafter, the components of the controlled-release formulation of the present invention are described in detail as follows:

1. Active Ingredient (Cilostazol)

In the controlled-release formulation of the present invention, cilostazol or a pharmaceutically acceptable salt thereof is used as an active ingredient. Cilostazol may be employed in an amount ranging from 10 to 80% by weight, preferably 30 to 50% by weight based on the total weight of the controlled-release formulation. When the amount is less than 10% by weight, the size of the formulation containing the recommended daily dose of cilostazol is 200 mg becomes too large to be orally administered to a patient, and when it is more than 80% by weight, the controlled release of the drug becomes cannot be achieved.

2. Solubilizing Agent

The inventive controlled-release formulation comprises a swelling agent which makes the formulation to float in the gastric juice in the stomach and intestines, which suppresses the release of poorly water-soluble cilostazol therein. Accordingly, a solubilizing carrier is included in the formulation to maintain a desired release rate of cilostazol.

The solubilizing agent may be at least one ingredients selected from the group consisting of polyvinylpyrrolidone, copovidone, polyethyleneglycol, hydroxyalkylcellulose, hydroxypropylmethylcellulose, poloxamer, polyvinylalcohol, cyclodextrin and a surfactant. The surfactant may include but are not limited to at least one ingredients selected from the group consisting of anionic surfactant, non-ionic surfactant, amphoteric surfactant or a mixture thereof, preferably poly(oxyethylene) sorbitan fatty acid ester, poly(oxyethylene) stearate, poly(oxyethylene) alkylether, polyglycolized glyceride, poly(oxyethylene) castor oil, sorbitan fatty acid ester, poloxamer, fatty acid salt, bile salt, alkylsulfate, lecithin, mixed micelles of bile salt and lecithin, sugar ester vitamin E(polyethylene glycol 1000)succinate (TPGS), sodium lauryl sulfate, and a mixture thereof.

The solubilizing agent may be employed in an amount ranging from 0.1 to 50% by weight, preferably 5 to 20% by weight based on the total weight of the inventive controlled-release formulation.

3. Swelling Agent

The controlled-release formulation of the present invention comprises a swelling agent capable of swelling the inventive formulation to extend its residence time in the stomach and intestines when orally administered, in order to enhance the absorption of cilostazol having restricted absorption sites.

Generally, the swelling agent is rapidly hydrated to absorb water when in contact with an external fluid or water, which allows the inventive formulation to swell and slowly release the drug. In the present invention, the swelling agent may be one of the known hydrophilic polymers, preferably hydrogels, which protect the drug from the surrounding condition as well as the partial accumulation of gases generated by the gas generating material, which allows the inventive formulation to float in the gastric fluid in the stomach and intestines.

Examples of swelling agents that can be used in the present invention include at least one selected from the group consisting of polyethyleneoxide, hydroxyalkylcellulose, hydroxypropylalkylcellulose, polyvinylalcohol, polyvinylpyrrolidone, sodium carboxymethylcellulose, carbopol, sodium alginate, xanthan gum, locust bean gum, cellulose gum, gellan gum, tragacanth gum, karaya gum, guar gum, and acacia gum. In the present invention, the polyethyleneoxide preferably has a molecular weight of 1,000,000 to 7,000,000, the hydroxyalkylcellulose is preferably hydroxyethylcellulose or hydroxypropylcellulose, and the hydroxypropylalkylcellulose is preferably hydroxypropylmethylcellulose.

The swelling agent may be employed in an amount ranging from 5 to 80% by weight, preferably 10 to 50% by weight, based on the total weight of the inventive controlled-release formulation.

4. Swell-Controlling Agent

Conventional swelling gastric-retention formulations exhibit unsatisfactory gastric retention or drug absorption due to its delayed swelling action after administration. In order to overcome the above problem, the present invention employs a swell-controlling agent to accelerate the formulation's swelling by the action of a swelling agent, which helps the formation to swell before the matrix breaks open, and also to promote the gas generation via the gas generating material by allowing the external fluid to rapidly infiltrate into the formulation. Accordingly, the controlled-release formulation of the present invention can satisfy the desired gastric retention time and drug absorption rate when orally administered.

The swell-controlling agent may be one of the swelling polymers, and representative examples thereof are cross-linked polyvinylpyrrolidone, cross-linked sodium carboxymethylcellulose, cross-linked calcium carboxymethylcellulose, cross-linked carboxymethylcellulose, sodium starch glycolate carboxymethyl starch, sodium carboxymethyl starch, potassium methacrylate-divinylbenzene copolymer, amylose, cross-linked amylose, starch derivative, microcrystalline cellulose, cellulose derivative, cyclodextrin, dextrin derivative, and a mixture thereof.

The swell-controlling agent may be employed in an amount ranging from 0.5 to 50% by weight, preferably 10 to 30% by weight, based on the total weight of the inventive controlled-release formulation.

5. Gas Generating Material

When the controlled-release formulation of the present invention is brought into contact with gastric juice, the gas generating material generates a gas on the surface and inside of the formulation, which allows the inventive formulation to float in the gastric juice of the stomach and intestines. Such floating of the inventive formulation occurs rapidly within 5 min after orally administered, which minimizes the problem of the controlled-release formulation passing the pylorus without achieving satisfactory swelling after its administration.

The gas generating material used in the present invention may be any one of the known gas generating materials which can generate a gas when contacted with gastric juice. Exemplary gas generating materials include at least one ingredients selected from the group consisting of uni- or bi-valent basic salts of carbonic acid (i.e., carbonates and bicarbonates) such as sodium hydrogen carbonate, sodium carbonate, sodium bicarbonate, potassium carbonate, potassium bicarbonate, calcium carbonate, magnesium carbonate and sodium glycine carbonate; and sulfites such as sodium sulfite, sodium bisulfite and sodium metabisulfite.

Further, the inventive controlled-release formulation may further comprise an acidic compound so that the gas generating material can generate a gas regardless of the surrounding pH. Such acidic compound may be an organic acid, an organic acid salt, or a mixture thereof, examples of which are citric acid, maleic acid, malic acid, fumaric acid, succinic acid, adipic acid, tartaric acid, malonic acid, monosodium citrate, ascorbic acid, glutamic acid, and a salt thereof.

The gas generating material may be employed in an amount ranging from 0.1% by weight to 50% by weight, preferably from 5% by weight to 20% by weight based on the total weight of the inventive controlled-release formulation.

6. Pharmaceutically Acceptable Additives

The controlled-release formulation of the present invention may further comprise pharmaceutically acceptable additives such as diluents, binding agents, lubricants, coating agents and plastisizers, in order to give the inventive formulation desirable properties in terms of, e.g., color, stability, controlled-release, suppression of burst release, and masking taste.

(1) Diluents

Examples of diluents are lactose, dextrin, mannitol, sorbitol, starch, microcrystallinecellulose, calcium hydrogen phosphate, anhydrous calcium hydrogen phosphate, calcium carbonate, sugars, and a mixture thereof.

(2) Binding Agents

Examples of binding agents are polyvinylpyrrolidone, copovidone, gelatin, starch, sucrose, methylcellulose, ethylcellulose, hydroxyethylcellulose, hydroxypropylcellulose, hydroxypropylalkylcellulose and a mixture thereof.

(3) Lubricants

Examples of lubricants are stearic acid, stearate, talc, corn starch, carnauba wax, light anhydrous silic acid, magnesium silicate, synthetic aluminum silicate, hardened oil, white lead, titanium oxide, microcrystallinecellulose, macrogol 4000 and 6000, isopropyl myristate, calcium hydrogen phosphate and a mixture thereof.

(4) Coating Agents

Examples of coating agents include at least one ingredients selected from the group consisting of ethylcellulose, shellac, ammonio methacrylate copolymer, polyvinylacetate, polyvinylpyrrolidone, polyvinylalcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose, hydroxyalkylcellulosephthalate, sodium celluloseacetatephthalate, celluloseacetylphthalate, celluloseetherphthalate, anionic copolymer of methacrylic acid and methyl or ethyl ester methacrylate, hydroxypropylmethylcellulose phthalate, hydroxypropylmethylcellulose acetylsuccinate, cellulose acetylphthalate and Opadry™ (Colorcon Co.), and exemplary ammonio methacrylate copolymers may include Eudragit RS™ or Eudragit RL™.

(5) Plastisizers

Examples of plastisizers include at least one ingredients selected from the group consisting of castor oil, fatty acid, substituted triglyceride and glyceride, triethylcitrate, polyethyleneglycol having a molecular weight of 300 to 50,000 and a derivative thereof.

The controlled-release formulation of the present invention may further comprise coloring agents, antioxidants, talc, titanic dioxide, flavoring agents and a mixture thereof.

The controlled-release formulation of the present invention may be prepared by a method comprising mixing cilostazol or a pharmaceutically acceptable salt thereof as an active ingredient, a solubilizing agent, a swelling agent, a swell-controlling agent, and a gas generating material and granulating the resulting mixture; and formulating the resulting mixture in the form of a capsule or a tablet.

Further, the controlled-release formulation of the present invention may be prepared by a method comprising mixing cilostazol and a solubilizing agent and granulating the resulting mixture; further mixing, or further mixing the resulting granules with a swelling agent, a swell-controlling agent and a gas generating material and granulating the resulting mixture; and formulating the resulting mixture into a capsule or a tablet.

In the above methods, the granulating step may be conducted through conventional granulation methods such as dry granulation, wet granulation, melt granulation, fluid bed granulation; direct compression; molding; and extrusion molding, preferably, fluid bed granulation, wet granulation, melt granulation and dry granulation, and two or more of the above methods may be used together in the present invention. Further, the granulating step may be conducted through a conventional wet granulating method, or a conventional solid dispersing method comprising a step of melting, dissolving by solvent, or refrigerating or drying after dispersing.

The above methods may further comprise the step of adding pharmaceutically acceptable additives such as binding agents and diluents to the mixture before performing the granulating process, or the step of adding pharmaceutically acceptable additives such as lubricants to the granule mixture after performing the granulating process.

Further, the tablet obtained in the above methods may be coated with a coating solution comprising a coating agent, a plastisizer or a mixture thereof in the present invention.

The coating agents or the binding agents may be employed as a solution prepared by dissolving them in water or an organic solvent, and the organic solvent may be methanol, ethanol, isopropanol, acetone, chloroform, chloromethane or a mixture thereof.

The following Examples are intended to further illustrate the present invention without limiting its scope.

### Example 1 and Comparative Examples 1 to 3

### Preparation of Matrix Tablets Comprising Cilostazol

Cilostazol matrix tablets of Example 1 and Comparative Examples 1 to 3 were prepared as follows, using the ingredients specified in Table 1.

TABLE 1

| | | Compar- | Compar- | Compar- |
|---|---|---|---|---|
| | Exam- | ative | ative | ative |
| Component | ple 1 | Example 1 | Example 2 | Example 3 |
| Cilostazol | 200 | 200 | 200 | 200 |
| Polyethyleneoxide | 150 | 150 | 150 | 200 |
| 5,000,000 | | | | |
| cross-linked sodium | 75 | 25 | 75 | 75 |
| carboxymethylcellulose | | | | |
| cross-linked | — | 25 | — | — |
| polyvinylpyrrolidone | | | | |
| Lactose | 27.5 | 50 | 27.5 | 27.5 |
| Sodium lauryl sulfate | 25 | 25 | 25 | 25 |
| hydroxypropylcellulose-L | 15 | 20 | 15 | 15 |
| light anhydrous silicic | 2.5 | — | 2.5 | 2.5 |
| acid | | | | |
| citric acid | 30 | — | — | — |
| sodium bicarbonate | 70 | — | — | — |
| magnesium stearate | 5 | 5 | 5 | 5 |
| Total weight (mg) | 600 | 500 | 500 | 550 |

A mixture of cilostazol, sodium lauryl sulfate, cross-linked sodium carboxymethylcellulose or cross-linked polyvinylpyrrolidone, and lactose was granulated, an ethanol solution of hydroxypropylcellulose-L was added thereto, and mixed. The resulting granules were fractionated using a 14 mesh, the particles that went through were dried, and further fractionated using an 18 mesh, and the granules passed through the mesh were mixed with: polyethyleneoxide (molecular weight: 5,000,000) and light anhydrous silicic acid (Comparative Examples 1 to 3); or polyethyleneoxide (molecular weight: 5,000,000), light anhydrous silicic acid, citric acid and sodium bicarbonate (Example 1). After adding magnesium stearate thereto, the resulting mixture was lubricated, and formulated into a tablet using a formulator, to obtain a controlled-release formulation. The controlled-release formulations of Example 1 and Comparative Examples 1 to 3 thus obtained are shown in Table 1.

### Test Example 1

### Dissolution Test

The matrix tablets obtained in Example 1 and Comparative Examples 1 to 3 were each subjected to a drug dissolution test using the USP dissolution test equipment. The time-dependent change of the drug dissolution rate was determined by the Paddle method conducted at 100 rpm/900 ml using a solution containing 0.5% sodium lauryl sulfate and 0.71% sodium chloride. The results are shown in Table 2.

TABLE 2

| | | Comparative | Comparative | Comparative |
|---|---|---|---|---|
| Time (hr) | Example 1 | Example 1 | Example 2 | Example 3 |
| 0 | 0.0 | 0.0 | 0.0 | 0.0 |
| 1 | 1.3 | 1.4 | 0.5 | 0.5 |
| 2 | 2.6 | 2.8 | 2.1 | 2.3 |
| 3 | 6.1 | 5.7 | 5.7 | 10.3 |
| 4 | 9.9 | 10.1 | 11.4 | 14.9 |
| 6 | 17.2 | 26.2 | 23.2 | 24.1 |
| 8 | 24.3 | 37.0 | 32.7 | 33.3 |
| 10 | 39.0 | 47.8 | 42.1 | 42.5 |
| 12 | 49.2 | 61.8 | 53.6 | 51.7 |
| 14 | 58.7 | 69.8 | 61.7 | 61.0 |
| 16 | 68.4 | 79.8 | 69.7 | 70.6 |

As shown in Table 2, the matrix of the tablet of Comparative Example 1 exhibited a slightly higher dissolution rate as compared to the matrixes of the tablets of Example 1, Comparative Example 2, and Comparative Example 3. 90% Drug release was observed at about 17 hours for the matrix tablet of Comparative Example 1, and the corresponding values for the tablets of Example 1, Comparative Example 2, and Comparative Example 3 were each about 20 hours. Further, the tablets of Example 1 and Comparative Examples 1 to 3 all exhibited similar controlled-release patterns with initial rates close to zero-order release.

This suggests that the drug release of the controlled-release pattern of the inventive formulation can be easily controlled by modifying the contents of the swelling agent, swell-controlling agent and gas generating material.

### Test Example 2

### Swelling Test

The matrix tablets obtained in Example 1 and Comparative Examples 1 to 3 were each subjected to a swelling or maintenance of swelling test with an USP dissolution test equipment. The time-dependent change of the tablet size was analyzed through picture-taking under the specified test conditions (artificial gastric juice of pH 1.2, Paddle method at 50 rpm/500 ml). The results are shown in Table 3.

TABLE 3

| | | | Comparative | | Comparative | | Comparative | |
|---|---|---|---|---|---|---|---|---|
| | Example 1 | | Example 1 | | Example 2 | | Example 3 | |
| | Major | Minor | Major | Minor | Major | Minor | Major | Minor |
| Time (hr) | axis | axis | axis | axis | axis | axis | axis | axis |
| 0 | 100.0 | 100.0 | 100.0 | 100.0 | 100.0 | 100.0 | 100.0 | 100.0 |
| 0.5 | 130.8 | 121.4 | 124.0 | 114.3 | 129.1 | 114.3 | 128.3 | 116.7 |
| 1 | 140.2 | 127.4 | 124.8 | 114.3 | 134.2 | 121.4 | 136.0 | 121.4 |
| 2 | 145.4 | 138.1 | 125.7 | 114.3 | 135.1 | 121.4 | 135.1 | 124.3 |
| 3 | 144.5 | 140.5 | 118.9 | 111.9 | 138.5 | 128.6 | 139.4 | 128.6 |
| 4 | 143.7 | 140.5 | 110.3 | 111.9 | 139.4 | 128.6 | 141.1 | 133.3 |
| 5 | 141.9 | 142.9 | 99.2 | 109.5 | 141.9 | 131.0 | 141.1 | 138.1 |
| 6 | 137.7 | 140.5 | 91.5 | 107.1 | 141.1 | 133.3 | 142.8 | 138.1 |
| 8 | 134.2 | 142.9 | 73.5 | 52.4 | 141.9 | 133.3 | 139.4 | 145.2 |
| 10 | 125.7 | 138.1 | 38.5 | 47.6 | 133.4 | 131.0 | 132.5 | 142.9 |
| 12 | 125.7 | 135.7 | 0.0 | 0.0 | 125.7 | 128.6 | 128.3 | 138.1 |

As shown in Table 3, the matrix tablet of Comparative Example 1 initially swelled somewhat but rapidly shrank after 2 hours, while the matrix tablets of Example 1, and Comparative Examples 2 and 3 expanded by more than 120% and maintained the expanded size even after 12 hours. The matrix tablet of Example 1, in particular, floated on the test solution within 5 min due to the action of the gas generation material component thereof. This shows an excellent initial-swelling behavior of the inventive controlled-release formulation.

The swelling or swelling-maintaining property of the controlled-release formulation can be maximized by adjusting the contents of the swelling agent, the swell-controlling agent and the gas generating material, especially the content of the gas generating material.

### Examples 2 to 11

### Preparation of Matrix Tablets Comprising Cilostazol

Cilostazol matrix tablets of Examples 2 to 11 were prepared as follows, using the ingredients specified in Tables 4a and 4b.

TABLE 4a

| | Exam- | Exam- | Exam- | Exam- | Exam- |
|---|---|---|---|---|---|
| Component | ple 2 | ple 3 | ple 4 | ple 5 | ple 6 |
| Cilostazol | 200.0 | 200.0 | 200.0 | 200.0 | 200.0 |
| Polyethyleneoxide | 150.0 | 150.0 | 150.0 | 150.0 | 150.0 |
| 5,000,000 | | | | | |
| cross-linked sodium | 123.8 | 75.0 | 75.0 | 172.5 | 91.2 |
| carboxymethylcellulose | | | | | |
| Lactose | 48.8 | 97.5 | 48.8 | 0.0 | 65.0 |
| Sodium lauryl sulfate | 25.0 | 25.0 | 25.0 | 25.0 | 25.0 |
| hydroxypropylcellulose- | 15.0 | 15.0 | 15.0 | 15.0 | 15.0 |
| L | | | | | |
| light anhydrous silicic | 2.5 | 2.5 | 2.5 | 2.5 | 2.5 |
| acid | | | | | |
| citric acid | 6.0 | 6.0 | 15.8 | 6.0 | 9.2 |
| sodium bicarbonate | 24.0 | 24.0 | 63.0 | 24.0 | 37.0 |
| magnesium stearate | 5.0 | 5.0 | 5.0 | 5.0 | 5.0 |
| Total weight (mg) | 600 | 600 | 600 | 600 | 600 |

TABLE 4b

| | Exam- | Exam- | Exam- | Exam- | Exam- |
|---|---|---|---|---|---|
| Component | ple 7 | ple 8 | ple 9 | ple 10 | ple 11 |
| Cilostazol | 200.0 | 200.0 | 200.0 | 200.0 | 200.0 |
| Polyethyleneoxide | 150.0 | 150.0 | 150.0 | 150.0 | 150.0 |
| 5,000,000 | | | | | |
| cross-linked sodium | 75.0 | 91.2 | 123.8 | 140.0 | 107.5 |
| carboxymethylcellulose | | | | | |
| Lactose | 0.0 | 16.2 | 0.0 | 16.2 | 32.5 |
| Sodium lauryl sulfate | 25.0 | 25.0 | 25.0 | 25.0 | 25.0 |
| hydroxypropylcellulose- | 15.0 | 15.0 | 15.0 | 15.0 | 15.0 |
| L | | | | | |
| light anhydrous silicic | 2.5 | 2.5 | 2.5 | 2.5 | 2.5 |
| acid | | | | | |
| citric acid | 25.5 | 19.0 | 15.8 | 9.2 | 12.5 |
| sodium bicarbonate | 102. | 76.0 | 63.0 | 37.0 | 50.0 |
| magnesium stearate | 5.0 | 5.0 | 5.0 | 5.0 | 5.0 |
| Total weight (mg) | 600 | 600 | 600 | 600 | 600 |

A mixture of cilostazol, sodium lauryl sulfate, cross-linked sodium carboxymethylcellulose or cross-linked polyvinylpyrrolidone, and lactose was granulated, an ethanol solution of hydroxypropylcellulose-L was added thereto, and mixed. The resulting granules were fractionated using a 14 mesh, the particles that went through were dried, and further fractionated using an 18 mesh, and the granules passed through the mesh were mixed with: polyethyleneoxide (molecular weight: 5,000,000), light anhydrous silicic acid, citric acid and sodium bicarbonate. After adding magnesium stearate thereto, the resulting mixture was lubricated, and formulated into a tablet using a formulator, to obtain a controlled-release formulation. The controlled-release formulations of Examples 2 to 11 thus obtained are shown in Tables 4a and 4b.

### Test Example 3

### Dissolution Test

The matrix tablets obtained in Examples 2 to 11 were each subjected to a drug dissolution test using the USP dissolution test equipment. The time-dependent change of the drug dissolution rate was determined under Paddle method conducted at 100 rpm/900 ml using a solution containing 0.5% sodium lauryl sulfate and 0.71% sodium chloride. The results are shown in Tables 5a and 5b.

TABLE 5a

| Time (hr) | Example 2 | Example 3 | Example 4 | Example 5 | Example 6 |
|---|---|---|---|---|---|
| 0 | 0.0 | 0.0 | 0.0 | 0.0 | 0.0 |
| 1 | 1.9 | 1.2 | 1.5 | 2.3 | 1.6 |
| 2 | 4.2 | 2.7 | 4.6 | 3.7 | 4.2 |
| 3 | 7.7 | 4.9 | 10.4 | 6.3 | 8.0 |
| 4 | 12.1 | 7.6 | 16.0 | 9.6 | 12.1 |
| 6 | 20.4 | 13.5 | 21.9 | 15.9 | 14.7 |
| 8 | 28.1 | 18.6 | 56.3 | 22.8 | 32.6 |
| 10 | 43.7 | 37.7 | 64.8 | 31.6 | 42.0 |
| 12 | 58.1 | 45.9 | 79.4 | 46.6 | 51.4 |
| 16 | 75.8 | 71.1 | 97.2 | 65.0 | 73.9 |
| 20 | 91.6 | 84.9 | 103.6 | 83.7 | 89.3 |

TABLE 5b

| | | | | | Example |
|---|---|---|---|---|---|
| Time (hr) | Example 7 | Example 8 | Example 9 | Example 10 | 11 |
| 0 | 0.0 | 0.0 | 0.0 | 0.0 | 0.0 |
| 1 | 2.2 | 1.6 | 2.2 | 1.8 | 2.1 |
| 2 | 6.4 | 4.8 | 5.3 | 3.6 | 4.9 |
| 3 | 11.2 | 9.3 | 10.0 | 6.5 | 8.7 |
| 4 | 16.7 | 12.2 | 13.6 | 9.2 | 13.3 |
| 6 | 29.1 | 20.8 | 26.4 | 22.7 | 20.3 |
| 8 | 41.2 | 28.0 | 38.4 | 31.8 | 34.7 |
| 10 | 53.0 | 37.0 | 49.0 | 40.8 | 44.4 |
| 12 | 73.7 | 64.5 | 64.2 | 50.8 | 59.5 |
| 16 | 89.0 | 82.1 | 82.9 | 69.3 | 75.0 |
| 20 | 101.5 | 95.1 | 98.7 | 85.7 | 90.1 |

As shown in Tables 5a and 5b, the controlled-release formulations of the present invention exhibited higher dissolution rate as the content of the gas generating material was increased, which results from that the internal structure of the matrix become loose to accelerate the infiltration of an external fluid. However, the contents of the swell-controlling agent and lactose did not affect the drug dissolution. Further, it was observed that all of the matrix tablets of Examples 2 to 11 floated on the test solution within 5 min and the floating was maintained until completion of test.

### Test Example 4

### Swelling Test

The matrix tablets obtained in Examples 2 to 11 were each subjected to a swelling test with an USP dissolution test equipment. The time-dependent change of the tablet size was analyzed through picture-taking under the specified test conditions (artificial gastric juice of pH 1.2, Paddle method at 50 rpm/500 ml). The results are shown in Tables 6a and 6b.

TABLE 6a

| | Example 2 | | Example 3 | | Example 4 | | Example 5 | | Example 6 | |
|---|---|---|---|---|---|---|---|---|---|---|
| Time | Major | Minor | Major | Minor | Major | Minor | Major | Minor | Major | Minor |
| (hr) | axis | axis | axis | axis | axis | axis | axis | axis | axis | axis |
| 0 | 100.0 | 100.0 | 100.0 | 100.0 | 100.0 | 100.0 | 100.0 | 100.0 | 100.0 | 100.0 |
| 0.5 | 131.6 | 116.7 | 130.7 | 114.3 | 129.8 | 114.3 | 130.7 | 114.8 | 131.6 | 114.8 |
| 1 | 138.6 | 119.0 | 137.7 | 119.8 | 140.4 | 121.4 | 139.5 | 121.4 | 139.5 | 121.4 |
| 2 | 144.7 | 127.4 | 146.5 | 128.6 | 143.0 | 127.4 | 145.6 | 128.6 | 142.1 | 131.0 |
| 3 | 144.7 | 133.3 | 147.4 | 135.7 | 143.0 | 133.3 | 148.2 | 133.3 | 143.0 | 133.3 |
| 4 | 146.5 | 135.7 | 150.9 | 141.0 | 143.9 | 138.1 | 150.0 | 139.3 | 143.9 | 139.3 |
| 5 | 144.7 | 135.7 | 150.9 | 142.9 | 138.6 | 138.1 | 151.8 | 140.5 | 143.9 | 139.3 |
| 6 | 142.1 | 136.9 | 147.4 | 141.7 | 136.0 | 135.7 | 150.9 | 138.1 | 143.0 | 135.7 |
| 8 | 136.8 | 135.0 | 144.7 | 142.9 | 133.3 | 131.0 | 145.6 | 138.1 | 140.4 | 133.3 |
| 10 | 129.8 | 132.1 | 137.7 | 140.5 | 126.3 | 131.0 | 143.9 | 140.5 | 130.7 | 132.1 |
| 12 | 125.4 | 132.1 | 128.1 | 131.0 | 119.3 | 129.8 | 135.1 | 138.1 | 125.4 | 123.8 |

TABLE 6b

| | Example 7 | | Example 8 | | Example 9 | | Example 10 | | Example 11 | |
|---|---|---|---|---|---|---|---|---|---|---|
| Time | Major | Minor | Major | Minor | Major | Minor | Major | Minor | Major | Minor |
| (hr) | axis | axis | axis | axis | axis | axis | axis | axis | axis | axis |
| 0 | 100.0 | 100.0 | 100.0 | 100.0 | 100.0 | 100.0 | 100.0 | 100.0 | 100.0 | 100.0 |
| 0.5 | 131.6 | 115.5 | 129.8 | 114.8 | 134.2 | 117.9 | 132.5 | 114.3 | 134.2 | 115.5 |
| 1 | 137.7 | 120.2 | 141.2 | 122.6 | 143.0 | 128.6 | 141.2 | 121.4 | 140.4 | 123.8 |
| 2 | 143.0 | 128.6 | 146.5 | 131.0 | 139.5 | 132.1 | 144.7 | 127.4 | 145.6 | 131.0 |
| 3 | 142.1 | 134.5 | 146.5 | 134.5 | 142.1 | 129.8 | 144.7 | 135.7 | 150.0 | 140.5 |
| 4 | 143.9 | 135.7 | 145.6 | 136.9 | 143.0 | 129.8 | 145.6 | 136.7 | 151.8 | 142.9 |
| 5 | 142.1 | 135.7 | 136.8 | 138.1 | 143.0 | 131.0 | 145.6 | 135.7 | 150.9 | 142.9 |
| 6 | 138.6 | 135.7 | 135.1 | 139.3 | 137.7 | 126.2 | 146.5 | 138.1 | 147.4 | 145.2 |
| 8 | 129.8 | 132.1 | 126.3 | 127.4 | 138.6 | 120.2 | 143.0 | 133.3 | 144.7 | 138.1 |
| 10 | 122.8 | 125.0 | 126.3 | 131.0 | 125.4 | 121.4 | 137.7 | 131.0 | 137.7 | 138.1 |
| 12 | 121.1 | 122.6 | 114.9 | 125.0 | 115.8 | 128.6 | 126.3 | 129.0 | 140.4 | 131.0 |

As shown in Table 6a and 6b, the swelling, especially initial swelling, of the inventive controlled-release formulations is affected by the adjustment of the relative contents of the swell-controlling agent, the gas generating material and the hydrophilic additive (lactose).

### Test Example 5

### Absorption Test

In order to determine the bioavailability of the drug contained in the inventive controlled-release formulation, the matrix tablet obtained in Example 11 and one of the commercially available tablet, Pletaal® (cilostazol 100, Otsuka Pharmaceutical Co., Ltd.), a comparative tablet, were respectively administered to beagle dogs (Beijing Marshall Biotechnology Co. Ltd., male, 5.5-week old, 6.94˜8.88 kg). Blood samples were taken from the dogs at regular intervals after the administration, and the time-dependent change of the blood drug concentration was analyzed. Based on the analyzed results, the maximum blood concentration (Cₘₐₓ), the time to reach the maximum blood concentration (Tₘₐₓ) and the area under the plasma concentration curve (AUC) of each tablet were calculated. The results are shown in Table 7.

TABLE 7

| | Cₘₐₓ (μg/ml) | Tₘₐₓ (hr) | AUC (ng * hr/ml) |
|---|---|---|---|
| Comparative tablet | 3.2 ± 1.45 | 2.33 ± 1.03 | 14.25 ± 10.41 |
| (100 mg * 1 time, n = 6) | | | |
| Example 11 | 4.67 ± 2.80 | 5.60 ± 2.19 | 24.13 ± 16.09 |
| (100 mg * 1 time, n = 6) | | | |

As shown in Table 7, the controlled-release formulation of the present invention exhibited a Tmax value which was more than twice that of the comparative tablet, which suggests that the inventive formulation exhibits a satisfactory sustained release pattern. Further, the controlled-release formulation of the present invention showed an AUC value which was higher than that of the comparative tablet by more than 170%. This suggests that the controlled-release formulation of the present invention provides a therapeutic effect which is twice that can be achieved by administering the comparative tablet.

Thus, the controlled-release formulation of the present invention can achieve extended gastric retention and sustained drug release characteristics.

As described above, the controlled-release formulation of the present invention has advantages in that it maintains a constant drug level of cilostazol in the blood through a slow release while it resides in the stomach and intestines over a long period of time, which maximizes the absorption of cilostazol in the small intestine known as the major absorption sites of cilostazol and as minimizes the previously mentioned problems associated with the conventional formulation.

While the invention has been described with respect to the above specific embodiments, it should be recognized that various modifications and changes may be made to the invention by those skilled in the art which also fall within the scope of the invention as defined by the appended claims.

What is claimed is:

1. A controlled-release formulation comprising cilostazol or a pharmaceutically acceptable salt thereof, a solubilizing agent, a swelling agent, a swell-controlling agent, and a gas generating material.

2. The controlled-release formulation of claim 1, which comprises cilostazol or the pharmaceutically acceptable salt thereof in an amount ranging from 10 to 80% by weight; the solubilizing agent in an amount ranging from 0.1 to 50% by weight; the swelling agent in an amount ranging from 5 to 80% by weight, the swell-controlling agent in an amount ranging from 0.5 to 50% by weight, and the gas generating material in an amount ranging from 0.1 to 50% by weight, based on the total weight of the formulation.

3. The controlled-release formulation of claim 1, wherein the solubilizing agent is selected from the group consisting of polyvinylpyrrolidone, copovidone, polyethyleneglycol, hydroxyalkylcellulose, hydroxypropylmethylcellulose, poloxamer, polyvinylalcohol, cyclodextrin, surfactant, and a mixture thereof.

4. The controlled-release formulation of claim 3, wherein the surfactant is selected from the group consisting of poly(oxyethylene)sorbitan fatty acid ester, poly(oxyethylene)stearate, poly(oxyethylene) alkyl ether, polyglycolized glyceride, poly(oxyethylene)castor oil, sorbitan fatty acid ester, poloxamer, fatty acid salt, bile salt, alkylsulfate, lecithin, mixed micelles of bile salt and lecithin, sugar ester vitamin E(polyethylene glycol 1000)succinate (TPGS), sodium lauryl sulfate, and a mixture thereof.

5. The controlled-release formulation of claim 1, wherein the swelling agent is selected from the group consisting of polyethyleneoxide, hydroxyalkylcellulose, hydroxypropylalkylcellulose, polyvinylalcohol, polyvinylpyrrolidone, sodium carboxymethylcellulose, carbopol, sodium alginate, xanthan gum, locust bean gum, cellulose gum, gellan gum, tragacanth gum, karaya gum, guar gum, acacia gum, and a mixture thereof.

6. The controlled-release formulation of claim 1, wherein the swell-controlling agent is selected from the group consisting of cross-linked polyvinylpyrrolidone, cross-linked sodium carboxymethylcellulose, cross-linked calcium carboxymethylcellulose, cross-linked carboxymethylcellulose, sodium starch glycolate, carboxymethyl starch, sodium carboxymethyl starch, potassium methacrylate-divinylbenzene copolymer, amylose, cross-linked amylose, starch derivative, microcrystallinecellulose and cellulose derivative, cyclodextrin and dextrin derivative, and a mixture thereof.

7. The controlled-release formulation of claim 1, wherein the gas generating material is selected from the group consisting of sodium hydrogen carbonate, sodium carbonate, sodium bicarbonate, potassium carbonate, potassium bicarbonate, calcium carbonate, magnesium carbonate and sodium glycine carbonate, sodium sulfite, sodium bisulfite, sodium metabisulfite, and a mixture thereof.

8. The controlled-release formulation of claim 7, wherein the gas generating material is used together with an acidic compound selected from the group consisting of an organic acid, an organic acid salt, and a mixture thereof.

9. The controlled-release formulation of claim 8, wherein the acidic compound is selected from the group consisting of citric acid, maleic acid, malic acid, fumaric acid, succinic acid, adipic acid, tartaric acid, malonic acid, monosodium citrate, ascorbic acid, glutamic acid, a salt thereof, and a mixture thereof.

10. The controlled-release formulation of claim 1, which further comprises pharmaceutically acceptable additives.

11. The controlled-release formulation of claim 10, wherein the pharmaceutically acceptable additives are selected from the group consisting of diluents, binding agents, lubricants, coating agents, plastisizers, and a mixture thereof.

12. The controlled-release formulation of claim 11, wherein the diluents are selected from the group consisting of lactose, dextrin, mannitol, sorbitol, starch, microcrystallinecellulose, calcium hydrogen phosphate, anhydrous calcium hydrogen phosphate, calcium carbonate, sugars, and a mixture thereof.

13. The controlled-release formulation of claim 11, wherein the binding agents are selected from the group consisting of polyvinylpyrrolidone, copovidone, gelatin, starch, sucrose, methylcellulose, ethylcellulose, hydroxyethylcellulose, hydroxypropylcellulose, hydroxypropylalkylcellulose, and a mixture thereof.

14. The controlled-release formulation of claim 11, wherein the lubricants are selected from the group consisting of stearic acid, stearate, talc, corn starch, carnauba wax, light anhydrous silicic acid, magnesium silicate, synthetic aluminum silicate, hardened oil, white lead, titanium oxide, microcrystallinecellulose, macrogol 4000 and 6000, isopropyl myristate and calcium hydrogen phosphate, sugars, and a mixture thereof.

15. The controlled-release formulation of claim 11, wherein the coating agents are selected from the group consisting of ethylcellulose, shellac, ammonio methacrylate copolymer, polyvinylacetate, polyvinylpyrrolidone, polyvinylalcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose, hydroxyalkylcellulosephthalate, sodium celluloseacetatephthalate, celluloseacetylphthalate, celluloseetherphthalate, anionic copolymer of methacrylic acid and methyl or ethyl ester methacrylate, hydroxypropylmethylcellulose phthalate, hydroxypropylmethylcellulose acetylsuccinate, cellulose acetylphthalate, Opadry, and a mixture thereof.

16. The controlled-release formulation of claim 11, wherein the plastisizers are selected from the group consisting of castor oil, fatty acid, substituted triglyceride and glyceride, triethylcitrate, polyethyleneglycol having a molecular weight of 300 to 50,000 and a derivative thereof, and a mixture thereof.

17. A method for preparing the controlled-release formulation of claim 1, which comprises the steps of mixing cilostazol or a pharmaceutically acceptable salt thereof, a solubilizing agent, a swelling agent, a swell-controlling agent and a gas generating material, granulating the resulting mixture, and formulating the resulting granule mixture in the form of a capsule or a tablet.

18. The method of claim 17, wherein the mixing and granulating step is conducted by mixing and granulating cilostazol or a pharmaceutically acceptable salt thereof and the solubilizing agent, further mixing the resulting granules with the swelling agent, the swell-controlling agent and the gas generating material, and granulating the resulting mixture.

19. The method of claim 17, which further comprises the step of adding binding agents and diluents to the mixture before performing the granulating process, or the step of adding lubricants to the granule mixture after performing the granulating process.

20. The method of claim 17, which further comprises the step of coating the tablet with a coating solution comprising a coating agent, a plastisizer or a mixture thereof.
